(19) United States
(12) Patent Application Publication
(10) Pub. No.: US 2008/0097277 A1
(43) Pub. Date: Apr. 24, 2008

(54) Controllable Shunt

(76) Inventor: Richard L. Saunders, Etna, NH (US)

Correspondence Address: MCANDREWS HELD & MALLOY, LTD, 500 WEST MADISON STREET, SUITE 3400, CHICAGO, IL, 60661, US

(21) Appl. No.: 11/815,809

(22) PCT Filed: Feb. 22, 2006
(86) PCT No.: PCT/US06/06081

§ 371 Date: Aug. 8, 2007

Related U.S. Application Data
(60) Provisional application No. 60/655,487, filed on Feb. 22, 2005.

(51) Int. Cl. A61M 5/00; A61M 1/00

(52) U.S. Cl. 604/9; 604/30; 604/31

(57) ABSTRACT

Apparatus and method for volumetric removal of cerebrospinal fluid (CSF) from the brain of patients suffering from hydrocephalus. The apparatus comprises a drainage conduit, which interconnects the cerebral ventricle system to a body cavity and an open and close pinch flow restrictor external to the drainage conduit to control flow. The flow restrictor has a controller that is remotely programmable. Also, the method provides for drainage of the CSF through the conduit by opening and closing the pinch flow restrictor for programmed durations. The actual opening and closing of the flow restrictor to actuate the drainage is determined by patient response to excessive or inadequate CSF diversion.

### CROSS-REFERENCE TO RELATED APPLICATION

This application claims the benefit of prior Provisional Application No. 60/655,487 filed on Feb. 22, 2005, the full disclosure of which is incorporated by reference.

### BACKGROUND OF THE INVENTION

This invention relates generally to surgically implanted medical equipment and methods for removing excess cerebrospinal fluid (CSF) from the brain for treatment of a condition known as hydrocephalus.

Hydrocephalus is an abnormal accumulation of CSF within cavities called ventricles inside the brain. Hydrocephalus occurs when there is an imbalance between the amount of CSF that is produced by the brain (about 20 ounces in 24 hours) and the rate at which it is absorbed. As the CSF builds up, it causes the ventricles to enlarge and, ultimately, the pressure inside the head to increase. The brain has the ability to absorb increases in volume of CSF, to a point, without a corresponding increase in intercranial pressure. The brain's ability to tolerate volume and pressure variations allows for CSF management systems that are not highly sensitive to variations of volume and pressure.

Historically, the management of hydrocephalus has taken two avenues: efforts to decrease CSF production with drugs or surgery or by assisting the evacuation or removal of excess fluid volume by the surgical placement of a shunt. The installation of a shunt is the most common method of treatment of hydrocephalus. A shunt is a flexible tube placed into the ventricular system that diverts the flow of CSF into another region of the body where it can be absorbed, such as the peritoneal (abdominal) cavity. The shunt tube is about ⅛ inch in diameter and is made of a soft and pliable silicone rubber extrusion that is well tolerated by body tissues. Generally, shunt systems come in a variety of models but have similar functional components. Components common to shunts include catheters (tubing) and a flow-control mechanism. Because elevated fluid pressure caused by an increase of CSF is inherent to symptomatic hydrocephalus, the standard flow control mechanism heretofore has been a pressure sensitive in-line valve. These pressure relief valves are designed to open at defined pressures and can be fixed pressure valves (available in different ranges) or adjustable (subsequent to implantation) pressure valves.

Shunt systems without integral resistances or in-line valves are less commonly used, but nevertheless acceptable when the inherent conduit resistance permits a flow of 20 ccs per minute on average. (Izurieta: Treatment of Hydrocephalus using an Open Ventricular Shunt in Adults. Presentation: Congress of Neurological Surgeons. San Diego; Oct. 1, 2001.)

Despite the wide use of pressure sensitive shunts, these shunts frequently prove problematic. In-line resistances inherent to these regulating valves increase the risk of obstruction by debris and/or bacteria. Valve blockage, under drainage and bacterial colonization require repeat surgery (known as “revision”) to replace the shunt system in approximately 40% of the cases. Additionally, revision may be necessary for overdrainage consequent to unnecessary fluid flow through pressure relief valves during normal transient pressure elevations from coughing or straining. Repeated surgery adds risk and cost to hydrocephalus management with current shunt technology.

Because of the brain's ability to tolerate variations in volume and transient extremes of CSF pressure, known as compliance, the use of pressure relief valves to address the disparity between CSF production and absorption represents undue system complexity.

For the foregoing reasons, there is a need for devices and methods for removing excess CSF from the brain such that the device is not disposed to the disadvantages of the prior art.

### SUMMARY OF THE INVENTION

It is a general object of the present invention to provide improved apparatus and methods for removing excess volume of CSF from the CSF space of a patient. The apparatus and methods of the present invention are particularly intended for the treatment of patients with an amount of CSF that is in excess of the CSF volume that can be naturally absorbed by the patient, and results in symptoms or raised intra-cranial pressure.

Another object of the invention is to provide a new and improved shunt system that is adapted to reduce system clogging by infected and non-infected debris.

Another object of the invention is to provide a new and improved method of managing the excess volume of CSF in the cerebral ventricular system by inserting a conduit and limiting the flow, arbitrarily, by periodic releases of CSF to create a balance between CSF production and absorption indifferent to moment to moment intra-cranial pressures. This method would rely on telemetric patient input to an implanted control system to direct intermittent conduit flow tailored to patient well being.

Another object of the invention is to provide apparatus and methods that do not depend on actual pressure or volume measurements of CSF for effective management of excess CSF production over absorption.

Another object of the invention is to provide a flow control system that, after implantation into the human body, can receive flow regulation instructions in a non-invasive manner.

Another object of the invention is to provide an intelligent system that can receive and retain actuating instructions remotely.

A further object of the invention is to provide a shunt system that includes separate implanted components such that failure of a component of the system would not require removal of the entire shunt system.

Another object of the invention is to provide an intelligent system that can send, receive, interpret, and respond to patient information and convey information through a remote programmer to an implanted control system.

The apparatus as disclosed and claimed herein comprises a shunt that will generally include an implantable compressible/closable conduit for draining CSF from the cerebral ventricular system into a body cavity, and a programmable flow control device to control CSF flow.

In a specific, preferred embodiment, the surgically implanted shunt will generally include an implanted conduit for draining symptomatic excess CSF from a patient's cerebral ventricles and depositing the fluid into a patient's body cavity, a flow restrictor external to the conduit that closes the conduit to restrict CSF flow through the conduit, an intelligent controller with memory capabilities for receiving and retaining instructions for actuating the flow restrictor and a remote intelligent programmer for input of patient symptoms and for transmitting to the controller open/closed flow restrictor instructions based on patient symptoms preferably through telemetry.

In a preferred embodiment the flow restrictor may be external to the conduit. One skilled in the art will realize alternative flow restrictors may be controlled by the controller of the present invention to obtain the desired CSF drainage flow of the present inventions.

At least one of the preceding objects is met, in whole or in part by the present invention. The aforesaid and other objects and advantages of the invention will become more apparent upon consideration of the preferred form of the shunt which is illustrated in the accompanying drawings wherein like parts are identified by the same numerals throughout the views.

### BRIEF DESCRIPTION OF THE DRAWINGS

FIG. 1 is a graph of the pressure volume relationship

FIG. 2 is an illustration of a ventriculo-peritoneal shunt.

FIG. 3 is a schematic of the components of one embodiment of the present invention.

FIG. 4 is an illustration one embodiment of a controller/flow restrictor assembly absent conduit.

FIG. 5 is an illustration of one embodiment of the controller/flow restrictor assembly with drainage conduit secured in place.

FIG. 6 is an illustration of a cross section of one embodiment of a flow restrictor for opening and closing drainage conduit shown in the closed position.

FIG. 7 is an illustration of a block diagram of the components of the remote programmer.

FIG. 8 is a flow chart illustrating the operation of the Controllable Shunt system

### DESCRIPTION OF THE PREFERRED EMBODIMENT OF THE INVENTION

As illustrated in the drawings, the volume shunt 10 is designed particularly for use in draining excess CSF fluid from the cerebral ventricular system and depositing it in a body cavity.

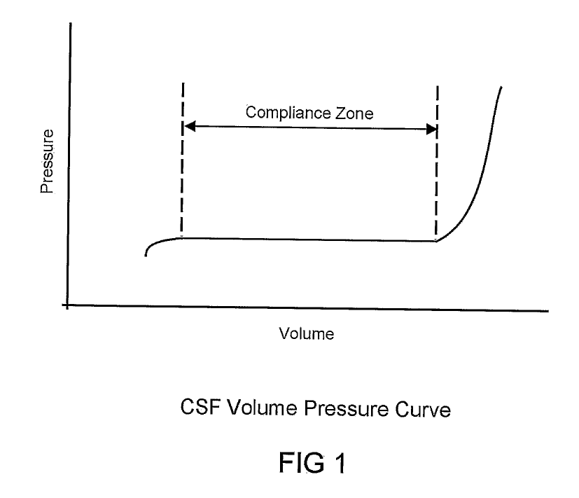

FIG. 1 illustrates the ability of the brain to tolerate increases of CSF without a corresponding increase in intercranial pressure. This tolerance, called compliance, affords the ability to design a CSF management system that does not depend on the measurement of CSF pressure or volume.

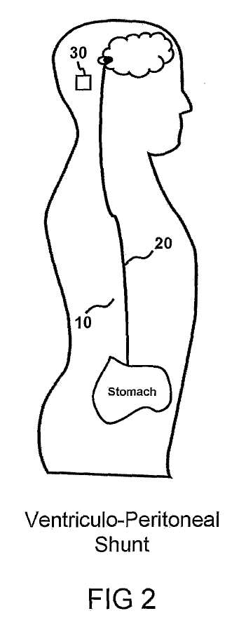

FIG. 2 illustrates a ventriculo-peritoneal shunt. The shunt 10 includes a drainage conduit 20 and a flow restrictor 30. This configuration is used by those skilled in the art and may be employed with the controllable shunt of the present invention.

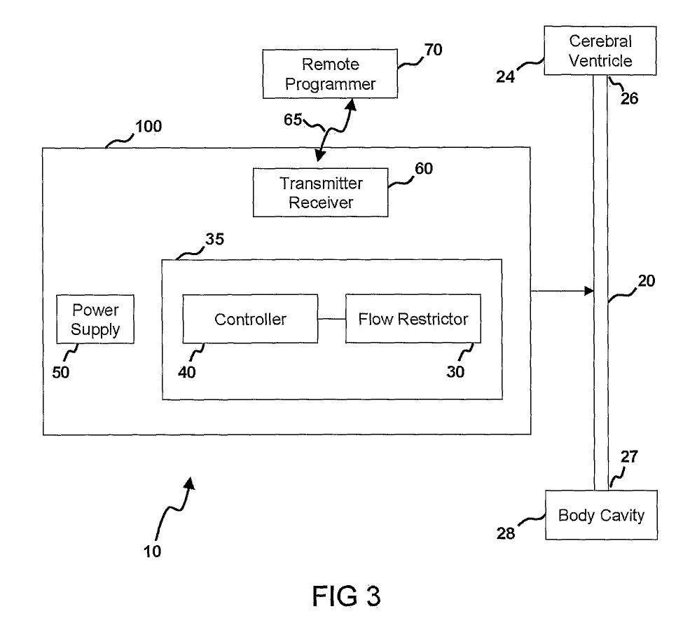

Referring to FIG. 3, a block diagram of the controllable shunt is indicated generally. Controllable shunt 10 preferably comprises a repeatably compressible/closable drainage conduit 20 and an intelligent control unit 100 that manages the flow of the excess CSF fluid. Control unit 100 and conduit 20 are made of a biocompatible material in order that they may be placed within a patient's body. The first end 26 of the drainage conduit 20 is positioned into the patient's cerebral ventricle 24. The second end 27 of the drainage conduit 20 is inserted into a patient's body cavity 28. Control unit 100 further comprises a controller/flow restrictor assembly 35, a power supply 50, and a transmitter/receiver 60. Controller/flow restrictor 35 generally is composed of a flow restrictor 30 and a microprocessor based controller 40. In one embodiment, implanted control unit 100 may measure approximately 3×3×1 cms. Persons skilled in the art will recognized that other dimensions are possible.

The present invention may employ a flow restrictor 30 of controller/flow restrictor assembly 35 that positively compresses/closes drainage conduit 20 to stop flow of CSF through conduit 20. Conduit 20 may be made of a biocompatible compressible silastic material or any other material that is biocompatible and tolerant to being repeatedly compressed.

Controller 40 utilizes power from power source 50. It is contemplated that power source 50 may be a battery similar to those used in pacemakers, defibrillators and the like. Power source 50 may be separate from controller/flow restrictor assembly 35 allowing replacement of power source 50 without replacing controller/flow restrictor assembly 35. It is contemplated that power source 50 would preferably be rechargeable using external energy. Power source 50 may also be a replaceable battery.

Remote programmer 70 may be an external computing device such as a lap top computer that wirelessly transmits commands to controller 40 to establish open/close periods for flow restrictor 30. Such flow restrictor 30 operation may be in response to individual patient symptoms caused by excessive or inadequate CSF diversion.

Controller 40 may be connected to a transmitter/receiver 60. The transmitter/receiver 60 receives open and close information from remote programmer 70 and transmits that information to controller 40. Transmitter/receiver 60 operates wirelessly preferably receiving a signal from remote programmer 70 by telemetry. Examples of telemetry systems that may include telemetry component adaptable for use with the present invention are shown in U.S. Pat. No. 5,683,432, U.S. Pat. No. 5,752,976, U.S. Pat. No. 5,843,139, and U.S. Pat. No. 5,904,708. In the present embodiment, it is contemplated that the signal is an RF signal.

Controller 40 of controller/flow restrictor assembly 35 may be any instrument that receives inputs from remote programmer 70 to open or close flow restrictor 30. It is contemplated that controller 40 is a digital system responsive to and programmable by remote programmer 70. Controller 40 may contain memory, control circuitry, timing circuitry to permit programmed operation of flow restrictor 30 based on instructions sent to controller 40 by remote programmer 70. Controller 40 may be programmed to operate flow restrictor 30 from instructions entered into remote programmer 70. It is contemplated that controller 40 will have the capabilities to retain open and close instructions for flow restrictor 30.

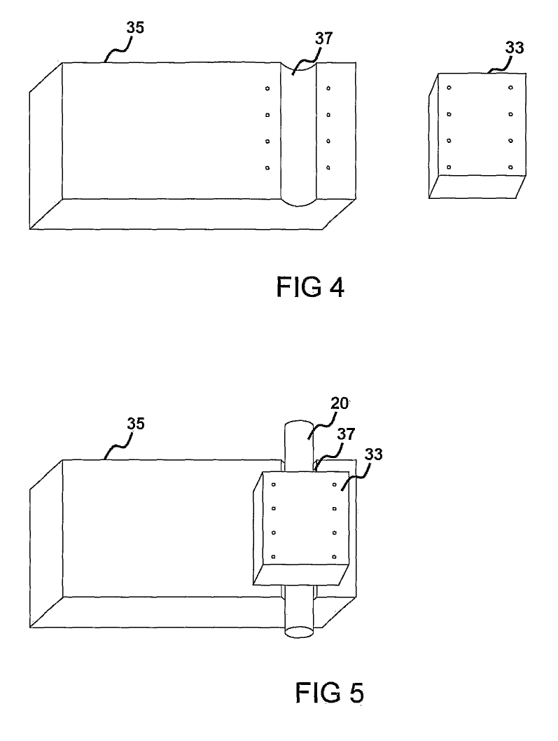

FIG. 4 is an illustration of an embodiment of controller/flow restrictor assembly 35 with channel slot 37 absent conduit 20 that is shown in FIGS. 3 and 5. FIG. 4 further illustrates retention plate 33. Retention plate 33 is shown detached from controller/flow restrictor assembly 35. Controller/flow restrictor assembly 35 has channel slot 37 in which conduit 20 (FIG. 3) would be positioned. Configuration of channel slot 37 would depend on the profile of conduit 20 (FIG. 3) and flow restrictor 30.

FIG. 5 is an illustration of controller/flow restrictor assembly 35 with conduit 20 in place. Conduit 20 passes through the flow restrictor assembly 35 via channel slot 37, but conduit 20 is not integral to flow restrictor assembly 35. This design creates no resistance to CSF flow through conduit 20 when flow restrictor assembly 35 is in the open position and a positive shutoff of conduit 20 when flow restrictor assembly 35 is in the closed position. FIG. 5 further illustrates retention plate 33 in place. Retention plate 33 may be made of silastic rubber or plastic (or other suitable biocompatible material) and secured to controller/flow restrictor assembly 35.

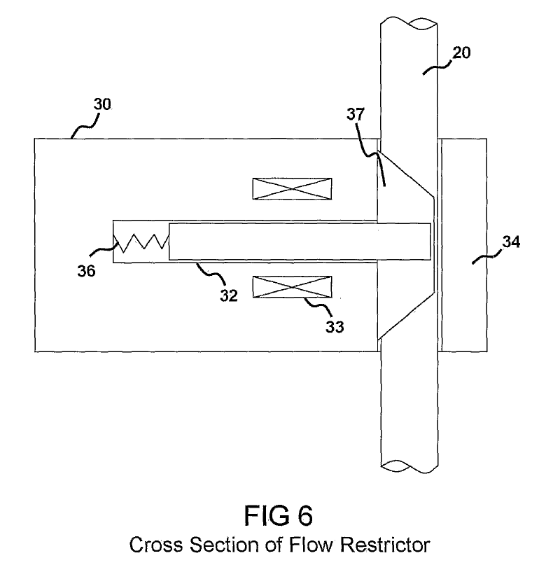

FIG. 6 is an exemplary cross section of an electrically controlled flow restrictor 30 shown in the closed position. Flow restrictor 30 is composed of a body 34 through which compressible/closable conduit 20 passes via channel slot 37. Flow restrictor body 34 comprises moveable element 32 and electromagnet 33 that is supplied with power from power source 50 (FIG. 3). Moveable element 32 engages and compresses conduit 20 against flow restrictor body 34 when electromagnet 33 is activated. A spring 36 is disposed between flow restrictor body 34 and moveable element 32 for biasing moveable element 32 away from conduit 20 allowing conduit 20 to be open when electromagnet 33 is not activated. When controller 40 (FIG. 3) sends a signal to energize electromagnet 33 of flow restrictor 30, moveable element 32 is extended to fully engage and compress conduit 20. Extension of moveable element 32 may be such that it presses conduit 20 against body 34 thus closing conduit 20 and preventing CSF flow through conduit 20. It is preferable that flow restrictor 30 would be normally open to allow flow of CSF through conduit 20 in the event of power failure of power supply 50 (FIG. 3).

Flow restrictor 30 is preferably normally in the open position to reduce power consumption from power supply 50 (FIG. 3) and thus extend the life of power supply 50. Electromagnet 33 would only be energized to extend moveable element 32 and close conduit 20 thus stopping flow of CSF.

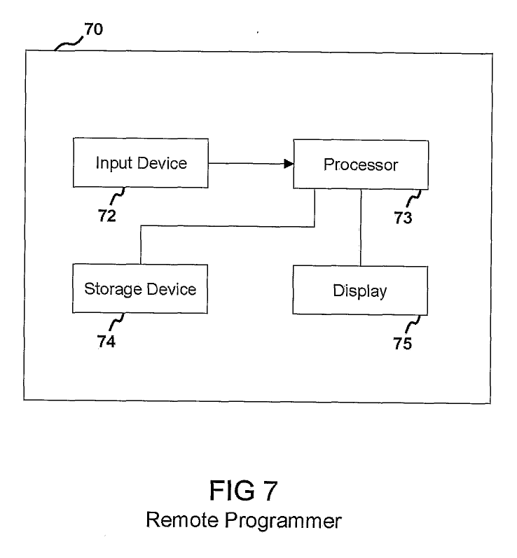

It is contemplated that flow restrictor 30 can be opened or closed for varying periods of time to manage CSF removal. In particular, the program of controller 40 (FIG. 3) of flow or no flow periods would be preferably communicated to controller 40 (FIG. 3) by remote programmer 70 (FIGS. 3 and 7) via telemetry. Alternatively, the flow restrictor 30 may be integral to conduit 20 as shown in FIGS. 5 and 6 may be managed by conventional volume or pressure controlled systems while still benefiting from some of the advantages of the present invention such as eliminating sites for clogging.

FIG. 7 is an illustration of an exemplary block diagram of remote programmer 70. Remote programmer 70 may include an input device 72, a processor unit 73, a storage device 74, and a display 75. Processing unit 73 may be a personal computer. Input device 72 may be a keyboard or a data port. Display 75 may be any computer display screen. Storage device 74 may be a hard disk drive. Instructions from remote programmer 70 may be transmitted to control unit 100 by telemetry. These instructions would be input into valve controller 40 for controlling the open/close status of flow restrictor 30.

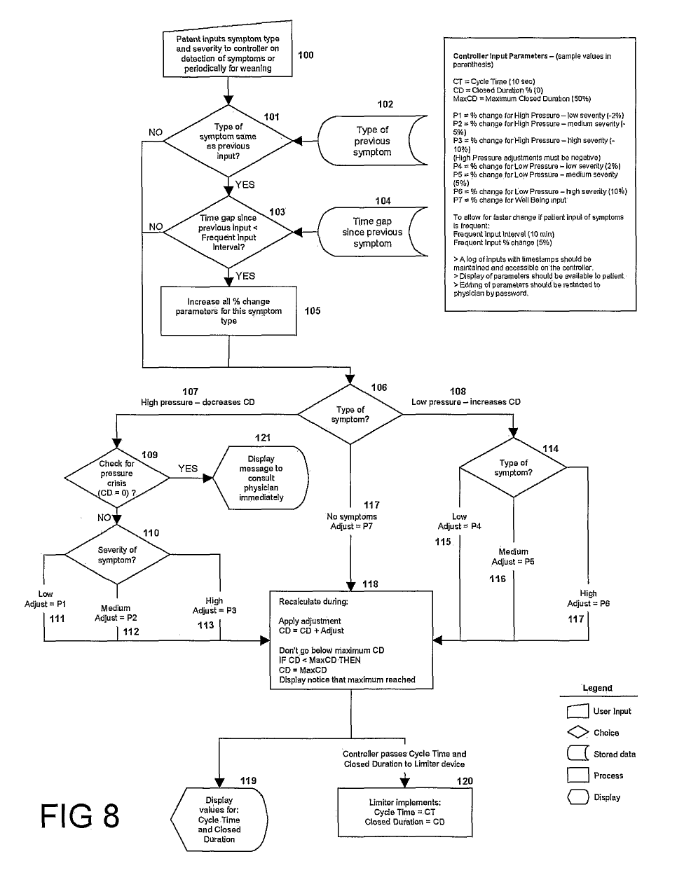

FIG. 8 illustrates a flow chart in accordance with an aspect of the invention. First, the user inputs data at user input block 100. Some of the data that may be entered at 100 are the type of symptom, that is high pressure headache or low pressure headache, and severity of the symptom, that is low, medium or high severity. Decision block 101 compares the type of symptom entered at 100 to the type of symptom previously entered, which is retained at stored data block 102. If at decision block 101 the type of symptom entered at block 100 is not the same as the symptom retained at data block 102, then at decision block 106 a comparison is made whether the symptom entered at block 100 is either high pressure symptom or low pressure symptom. If at decision block 101, the symptom entered at block 100 is the same as the symptom as stored in stored data block 102, then at decision block 103, the time since the previous input, retained at stored data block 104, is compared with a predetermined frequent input interval. If the time since last symptom entry at block 100 is greater than the frequent input interval, then at decision block 106, a comparison is made whether the symptom entered at block 100 is either high pressure symptom or low pressure symptom. If at decision block 103, the time since the last symptom input in stored data block 104 is less than the predetermined frequent input interval, then process block 105 increases all the percent change parameters for the type of symptom entered at block 100 and decision block 106 compares whether the symptom input at block 100 is either high pressure symptom or low pressure symptom.

If at decision block 106, the symptom entered at block 100 is high pressure, then at decision block 109, the closed duration (CD) of valve is checked to see if it is zero. If zero, a message to consult physician is displayed at display block 121. If the closed duration is not zero then decision block 110 compares whether the severity of the symptom entered at block 100 is low, medium, or high severity. If the severity of the high pressure symptom entered at block 100 is low severity, then the valve closed duration (CD) is changed by predetermined percent 111. If the severity of the high pressure symptom entered at block 100 is medium severity, then the valve closed duration (CD) is changed by predetermined percent 112. If the severity of the high pressure symptom entered at block 100 is high severity, then the valve closed duration (CD) is changed by predetermined percent 113.

If at decision block 106, no symptom is entered at block 100, then closed duration (CD) is adjusted by 117.

If at decision block 106, the symptom entered at block 100 is a low pressure symptom, then decision block 114 compares whether the severity of the symptom entered at block 100 is low, medium, or high severity. If the severity of the low pressure symptom entered at block 100 is low severity, then the valve closed duration (CD) is changed by predetermined percent 115. If the severity of the symptom entered at block 100 is medium severity, then the valve closed duration (CD) is changed by predetermined percent 116. If the severity of the symptom entered at block 100 is high severity, then the valve closed duration (CD) is changed by predetermined percent 117. It will be appreciated that the percentage parameters are arbitrary and can be modified through clinical experience.

After evaluation of symptoms at either decision blocks 110, 114, or 117, process block 118 recalculates duration (CD) and display block 119 displays new values for cycle time (CT) and closed duration (CD). At process block 120, the cycle time (CT) and closed duration (CD) are sent to controller 40 (FIG. 3) to activate flow restrictor 30 (FIG. 3).

It is further contemplated that the invention, in one embodiment, includes a method for creating a balance of CSF in a patient diagnosed with hydrocephalus. The method includes installing a CSF drainage device 20 and modulating flow restrictor 30 to control CSF flow through conduit 20. It will be appreciated that the period of flow or no flow of CSF through conduit 20 would be based on the patient's symptoms of over-drainage or under-drainage. The opening and closing of flow restrictor 30 may occur many times in any period tailored to the individual patient's requirements. The period may be any time period, but it is initially contemplated the patient's systems would be evaluated over a several day period.

In one example, device 10 is installed with flow restrictor 30 in the open position to allow for continuous drainage of CSF through conduit 20. It will be appreciated that the patient and attendants will be continuously monitoring for symptoms of over-drainage as the patient recovers from the shunting procedure. This open position will allow tailoring controller 40 to the patient's CSF diversion requirements. Determination of over-drainage during this calibration period may lead to flow restrictor 30 activation via instructions entered into remote programmer 70 and transmitted via telemetry 65 to flow restrictor controller 40. Flow restrictor controller 40 would then open or close flow restrictor 30 based on the instructions entered into remote programmer 70. It is contemplated that during this first set of adjustments, flow restrictor 30 would be set to open for a cumulative time of ⅓ of a unit of time. The opening or closing of flow restrictor 30 may be programmed to occur many times during the 24 hour period with a cumulative ⅓ open time period. Persisting symptoms of inadequate balance of CSF may prompt a second flow restrictor 30 adjustment to longer cumulative periods of flow restrictor 30 open or close status. It will be appreciated that adjustments to flow restrictor 30 may be made until patient's symptoms indicate a balance has been achieved between the production and adsorption of CSF. This protocol may be individualized to each patient and controller 40 would retain the flow restrictor 30 open and closing periods.

Another iteration may be three or more preprogrammed controller/flow restrictor assembly 35 24-hour programs, which in a unit of time may see the flow restrictor 30 open ⅓, ½ or ⅔ of the time.

In another example, flow restrictor 30 may be instructed to remain open through an extended portion of the selected time period, and then resume a pre-selected modulation during the remainder of the time period. A specific example may be that instructions would be entered into remote programmer 70 to be transmitted via telemetry 65 to controller 40 that flow restrictor 30 would remain open when the patient is sleeping and then instructed to resume the pre-selected flow restrictor 30 modulation for the remainder of the period once the patent is awake. It will be appreciated that these instructions may be preprogrammed for the entire period or selectively altered depending on the individual patient's activity.

In another example, a method of weaning from shunt 10 dependence is possible with the present invention. A weaning mode would involve the programming of the programmable flow restrictor 35 through the remote programmer 70 to allow for open/close periods involving small incremental decreases in the open time of conduit 20 through the course of 24 hours. Continued regular inputs of well being input into the remote programmer 70 would permit the continued decreasing of open time of conduit 20 beyond 24 hours. The decreasing of the amount of open time of conduit 20 would continue as long as no CSF imbalance symptom is entered into the remote programmer 70. Regular inputs of well being into remote programmer 70 would allow continuation of decreasing the time of open conduit 20 until conduit 20 is closed one hundred percent of the time. This method would be applicable to patients who have protracted well being over extended periods with no necessity of new programming. It will be appreciated that exact periods of decreasing open time for this method would be determined by the managing physician and would be made over an extended period of time.

It is further contemplated that Computed Axial Tomography (CT scans) of the cerebral ventricular system may be used to assist in identifying over-drainage or under-drainage.

Initially, it is preferable that the physician would determine the time and frequency of remote programmer 70 input to actuate open or close status of flow restrictor 30. After counseling of the protocol by the physician, the individual patient may modify the behavior of flow restrictor 30 based on the patient's own perception of over-drainage or under-drainage symptoms, and thereby teach the system from individual patterns of drainage and their effect on well being.

Although it is preferred to use an integral conduit 20 such as shown in FIGS. 5 and 6 with symptom-driven controller 40 of the present invention, in-line conventional flow restrictors used within the shunting passage of prior art devices may also be used in the present invention while still benefiting from some of the advantages of the present invention provided by the symptom driven controller.

While the invention has been described with reference to preferred embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed, but that the invention will include all embodiments falling within the scope of the appended claims.

What is claimed is:

1. An apparatus for draining cerebrospinal fluid (CSF) from a patient's cerebral ventricle system and depositing the CSF into a body absorption site comprising: a passage capable of shunting CSF from the patient's cerebral ventricle system to the body's absorption site; a control unit for managing CSF flow based on patient symptoms; and a remote programmer adapted to communicate operating instructions to the control unit.

2. The apparatus of claim 1 wherein the patient's body absorption site is a patient's peritoneal cavity.

3. The apparatus of claim 1 wherein the control unit further comprises a flow restrictor.

4. The apparatus of claim 3 wherein the flow restrictor comprises a movable closing element.

5. The apparatus of claim 3 wherein the control unit comprises an in-line flow restrictor.

6. The apparatus of claim 3 wherein the control unit further comprises a microprocessor based controller, wherein the controller operates the flow restrictor.

7. The apparatus of claim 6 wherein the controller is remotely programmable to operate the flow restrictor based on patient symptoms.

8. The apparatus of claim 6 wherein the controller is capable of retaining preprogrammed operating instructions.

9. The apparatus of claim of claim 1 wherein the control unit further comprises a transmitter/receiver for receiving flow restrictor operating instructions from the remote programmer.

10. The apparatus of claim 1 wherein the remote programmer wirelessly transmits operating instructions to the control unit.

11. A method of controlling flow of CSF fluid comprising: identifying a patient with hydrocephalus; implanting in the patient a conduit for establishing drainage of CSF fluid from a patient's cerebral ventricular system to a patient's body cavity; implanting in the patient a programmable flow restrictor; and controlling a patient's CSF flow by restricting flow through the conduit using the flow restrictor based on a patient's symptoms of inadequate or excessive CSF drainage.

12. A method as in claim 11 wherein the patient's symptom of inadequate CSF drainage is a headache, especially in the morning, possibly associated with visual problems and nausea.

13. A method of claim 11 wherein the patient's symptom of excessive CSF drainage is a low-pressure headache relieved by reclining and not associated with neurological impairment.

14. A method of claim 11 wherein controlling CSF flow comprises opening and closing the implanted flow restrictor based on input of patient symptoms to remote programmer.

15. A method as in claim 14 wherein the flow restrictor is opened for a predetermined time period.

16. A method as in claim 14 wherein the flow restrictor is opened and closed at pre-selected times during the predetermined period determined by the patient's symptoms of inadequate or excessive CSF drainage.

17. A method as in claim 14 wherein the flow restrictor is opened for serially decreasing durations during a period when there are no patient inputs of inadequate or excessive CSF drainage.

18. An apparatus for draining cerebrospinal fluid (CSF) from a patient's cerebral ventricles and depositing the CSF into a body absorption site comprising: a conduit with a first end inserted into the patient's cerebral ventricle and a second end inserted into a patient's body absorption site; a flow restrictor to control CSF flow based on a patient's symptoms; a control unit for operating flow restrictor; and a remote programmer adapted to communicate operating instructions to the control unit.

19. The apparatus in claim 18 wherein the open and closed status of the flow restrictor is based on the patient symptoms.

20. The apparatus in claim 18 wherein flow restrictor engages the conduit between the first and second ends to control CSF flow.

21. The apparatus in claim 18 wherein the controller is programmable to allow different periods of opening and closing of the flow restrictor.

22. The apparatus in claim 18 wherein the controller is capable of retaining preprogrammed operating instructions.
